(19) United States
(12) Patent Application Publication
(10) Pub. No.: US 2005/0244508 A1
(43) Pub. Date: Nov. 3, 2005

(54) Anti-spasmodic comprising xenon

(75) Inventors: Peter Neu, Duisburg (DE); Carsten Pilger, Rheurdt (DE); Matthias Reyle-Hahn, Berlin (DE)

Correspondence Address: CONNOLLY BOVE LODGE & HUTZ, LLP, P O BOX 2207, WILMINGTON, DE, 19899, US

(73) Assignee: Messer Griesheim

(21) Appl. No.: 10/517,722

(22) PCT Filed: Jun. 12, 2003
(86) PCT No.: PCT/EP03/06190

§ 371 Date: Dec. 10, 2004

(30) Foreign Application Priority Data
Jun. 12, 2002 (DE) DE 102 26 191.1
Jun. 12, 2002 (DE) DE 102 26 193.8
Jun. 22, 2002 (DE) DE 102 27 974.8

(51) Int. Cl. A61K 33/00

(52) U.S. Cl. 424/600; 424/718

(57) ABSTRACT

Xenon or xenon-containing gases and, where appropriate, an NO source are employed as spasmolytic. The spasmolytic is preferably a medicament for the treatment of vasospasms, in particular for the treatment of cerebral vasospasms or coronary vasospasms.

The invention relates to a medicament comprising xenon.

WO 02/22141 A2 describes the use of xenon or xenon-containing gases as medicament, in particular cardiovascular agent.

Spasmolytics include medicaments which reduce the tone of smooth muscles (gastrointestinal tract, vessels, bronchi).

The invention is based on the object of providing an alternative medicament, in particular an alternative spasmolytic.

The invention relates to a spasmolytic having the features described in claim 1.

The spasmolytic is a substance or mixture of substances comprising xenon. The spasmolytic preferably consists of gaseous xenon or a xenon-containing gas mixture which is normally administered by inhalation. The spasmolytic generally acts on smooth muscles, especially on the smooth muscles of vessels. The spasmolytic is generally a spasmolytic of smooth muscles.

The spasmolytic is preferably a medicament for the treatment of vasospasms, in particular for the treatment of cerebral vasospasms or coronary vasospasms.

The invention thus further relates to the use of xenon or a xenon-containing gas as vasodilator, preferably as capillary or precapillary vasodilator, in particular as vasodilator in capillary or precapillary vascular systems of the human body.

The invention further relates to the use of xenon or a xenon-containing gas for producing a medicament for vasodilatation, preferably for producing a medicament for capillary or precapillary vasodilatation, in particular for producing a medicament for vasodilatation in capillary or precapillary vascular systems of the human body.

The spasmolytic is preferably gaseous. It preferably comprises no solid or liquid constituents on administration. The spasmolytic is thus preferably in the form of a pure gas phase on administration. The spasmolytic comprises xenon in a pharmacologically or therapeutically effective amount, in particular in spasmolytically effective amounts. The spasmolytic is ordinarily a xenon-containing gas mixture with a content of at least 1% by volume xenon. The spasmolytic is preferably administered by inhalation through the lungs. In this case, the spasmolytic is an inhaled spasmolytic. The spasmolytic is also administered by means of a heart-lung machine. The spasmolytic is preferably used for treating humans.

The spasmolytic is normally provided as pure gaseous xenon. The spasmolytic can also be provided as gas mixture. The spasmolytic is normally employed as a gas mixture which maintains respiration and comprises xenon and oxygen. Such gas mixtures are employed for example in emergency medicine, where gas-mixing or gas-metering devices are too complicated for mobile use.

Gaseous xenon or xenon-containing gas mixtures are particularly advantageously employed for the prophylaxis of spasms, preferably vasospasms. Prophylactic administration of xenon or xenon-containing gas mixtures takes place for example preoperatively, intraoperatively or postoperatively.

The provided spasmolytic or the spasmolytic produced directly on use, in particular in the direct vicinity of the patient, is for example a gas mixture which comprises from 1 to 80% by volume (based on standard conditions, i.e. 20° C., 1 bar absolute) of xenon (e.g. remainder oxygen). The spasmolytic administered to the patient preferably comprises xenon in amounts which have no anesthetic effect. Xenon amounts having no anesthetic effect or being inadequate for anesthesia are referred to as subanesthetic or subanesthetically effective amounts. Gas mixtures with contents of up to 70% by volume xenon generally contain subanesthetic amounts of xenon. Gas mixtures administered as spasmolytic preferably comprise up to 65% by volume, particularly preferably up to 60% by volume, in particular up to 50% by volume, xenon. For example, pure xenon is accordingly metered into a patient's respiratory gas in such a way that gas mixtures with said xenon concentrations are produced. This means that the respiratory gas which has been produced and supplied to the patient comprises, for example, from 5 to 60% by volume, 5 to 50% by volume, 5 to 40% by volume, 5 to 30% by volume or 5 to 20% by volume xenon. In special cases, e.g. in the prophylaxis of spasms, especially on prolonged ventilation, it may be advantageous to meter xenon in the respiratory gas with a low concentration, for example from 1 to 35% by volume, 5 to 25% by volume or 5 to 20% by volume or 5 to 10% by volume xenon in the respiratory gas.

It may be worthwhile in emergency situations to administer xenon as spasmolytic in high concentration.

The gas mixtures administered as spasmolytic preferably comprise besides xenon one or more gases or substances which are gaseous at body temperature under atmospheric pressure. Examples of such gas mixtures are xenon-oxygen gas mixtures or gas mixtures of xenon and one or more inert gases such as nitrogen or a rare gas or xenon-oxygen-inert gas mixtures. Admixture of a gas, in particular of an inert gas, may be very advantageous if it is intended to introduce little xenon into the body. Examples of gases or gas mixtures which are employed as spasmolytic, in particular as vasospasmolytic, follow: 1.) 100% by volume xenon; 2.) 70% by volume xenon/30% by volume oxygen; 3.) 65% by volume xenon/30% by volume oxygen/5% by volume nitrogen; 4.) 65% by volume xenon/35% by volume oxygen; 5.) 60% by volume xenon/30% by volume oxygen/10% by volume nitrogen; 6.) 60% by volume xenon/35% by volume oxygen/5% by volume nitrogen; 7.) 60% by volume xenon/40% by volume oxygen; 8.) 55% by volume xenon/25% by volume oxygen/20% by volume nitrogen; 9.) 55% by volume xenon/30% by volume oxygen/15% by volume nitrogen; 10.) 55% by volume xenon/35% by volume oxygen/10% by volume nitrogen; 11.) 55% by volume xenon/40% by volume oxygen/5% by volume nitrogen; 12.) 55% by volume xenon/45% by volume oxygen; 13.) 50% by volume xenon/50% by volume oxygen; 14.) 50% by volume xenon/45% by volume oxygen/5% by volume nitrogen; 15.) 50% by volume xenon/40% by volume oxygen/10% by volume nitrogen; 16.) 50% by volume xenon/30% by volume oxygen/20% by volume nitrogen; 17.) 50% by volume xenon/25% by volume oxygen/25% by volume nitrogen; 18.) 45% by volume xenon/55% by volume oxygen; 19.) 45% by volume xenon/50% by volume oxygen/5% by volume nitrogen; 20.) 45% by volume xenon/45% by volume oxygen/10% by volume nitrogen; 21.) 45% by volume xenon/40% by volume oxygen/15% by volume nitrogen; 22.) 45% by volume xenon/35% by volume oxygen/20% by volume nitrogen; 23.) 45% by volume xenon/30% by volume oxygen/25% by volume nitrogen; 24.) 45% by volume xenon/30% by volume oxygen/25% by volume nitrogen; 25.) 40% by volume xenon/30% by volume oxygen/30% by volume nitrogen; 26.) 40% by volume xenon/50% by volume oxygen/10% by volume nitrogen; 27.) 35% by volume xenon/25% by volume oxygen/40% by volume nitrogen; 28.) 35% by volume xenon/65% by volume oxygen; 29.) 30% by volume xenon/70% by volume oxygen; 30.) 30% by volume xenon/50% by volume oxygen/20% by volume nitrogen; 31.) 30% by volume xenon/30% by volume oxygen/40% by volume nitrogen; 32.) 20% by volume xenon/80% by volume oxygen; 33.) 20% by volume xenon/30% by volume oxygen/50% by volume nitrogen; 34.) 15% by volume xenon/30% by volume oxygen/55% by volume nitrogen; 35.) 15% by volume xenon/50% by volume oxygen/35% by volume nitrogen; 36.) 10% by volume xenon/90% by volume oxygen; 37.) 10% by volume xenon/50% by volume oxygen/40% by volume nitrogen; 38.) 10% by volume xenon/30% by volume oxygen/60% by volume nitrogen; 39.) 10% by volume xenon/25% by volume oxygen/65% by volume nitrogen; 40.) 5% by volume xenon/25% by volume oxygen/70% by volume nitrogen; 41.) 5% by volume xenon/30% by volume oxygen/65% by volume nitrogen; 42.) 5% by volume xenon/50% by volume oxygen/45% by volume nitrogen; 43.) 5% by volume xenon/30% by volume oxygen/65% by volume nitrogen; 44.) 5% by volume xenon/95% by volume oxygen; 45.) 1% by volume xenon/99% by volume oxygen; 46.) 1% by volume xenon/30% by volume oxygen/69% by volume nitrogen; 47.) 1% by volume xenon/25% by volume oxygen/74% by volume nitrogen.

Xenon or a xenon-containing gas mixture is preferably used to produce a medicament for the treatment of spasms, to produce a medicament for the treatment of vasospasms, to produce a medicament for the treatment of vasospasms in capillary vascular systems (e.g. vasospasms of capillary vessels) or precapillary vascular systems.

Medicaments for the treatment of cerebral vasospasms which comprise xenon are referred to as cerebrospasmolytics. The cerebrospasmolytics counteract impairments of blood flow in the brain. The cerebrospasmolytics are further used for the treatment of impairment of cerebral perfusion and of cognitive impairments. The cerebrospasmolytics are additionally employed for the prophylaxis and/or therapy of impairments of cognitive performance, also postoperatively. The cerebrospasmolytics are used for the treatment of stroke and for the propylaxis of stroke. The cerebrospasmolytics are further used for the treatment of post-ischemia syndromes.

In addition, xenon or a xenon-containing gas mixture are also used to produce a bronchospasmolytic.

In addition, xenon or a xenon-containing gas mixture are also used to produce a vasospasmolytic for the treatment of coronary perfusion impairments.

Said medicaments are regarded as special forms of a spasmolytic. The term “spasmolytic” is the general term covering the particular medicaments mentioned. The statements about the composition and administration of the spasmalytic are therefore applicable to the particular medicaments.

The spasmolytic and the particular medicaments are employed preoperatively, intraoperatively or postoperatively.

The spasmolytic is particularly advantageously employed in intensive-care medicine, especially when the medicament must be administered over a prolonged period, for example during long-term ventilation. In this case, the spasmolytic has the particular advantage of having no side effects according to the current state of knowledge. When xenon or xenon-containing gases are used as spasmolytic there is no formation of metabolites in the body, and the medicament does not accumulate in the body.

Xenon is administered in particular during long-term ventilation and for prophylaxis in subanesthetic concentrations in the breathable gas (respiratory gas). It is advantageous especially during long-term ventilation to administer breathable gases having a content of from 5 to 45% by volume xenon, preferably 5 to 40% by volume xenon. During long-term ventilation, the breathable gas has, for example, a content of from 20 to 30% by volume oxygen, it being possible to increase the oxygen content if required at times for example 30 to 95% by volume oxygen. The remaining gas in the breathable gas usually consists of nitrogen or another inert gas.

Xenon- and oxygen-containing gas mixtures are advantageously employed as bronchospasmolytic in particular in the homecare sector for supplying oxygen (e.g. long-term oxygen therapy, especially for asthma or COPD (chronic obstructive pulmonary disease) or for acute respiratory distress) of spontaneously breathing patients. The xenon- and oxygen-containing gas mixtures have for example a xenon content in the range from 1 to 30% by volume xenon, preferably in the range from 1 to 20% by volume xenon, in particular in the range from 5 to 10% by volume xenon. The gas mixture is provided in compressed gas containers or as cryogenically liquefied gas in insulated containers or is generated on site.

The employed xenon gas generally has the natural isotope composition. The isotope composition of the xenon may differ from the natural isotope composition, in particular on use for diagnostic purposes. The xenon gas is preferably employed in high purity, as usual for medical gases. The xenon gas is preferably used as pure gas or mixed with other gases for producing a gaseous medicament for said applications.

Gaseous xenon (pure xenon) is generally provided as compressed gas in compressed gas containers such as compressed gas cylinders or pressurized cans. It is also possible to provide xenon-containing gas mixtures in compressed gas containers. The gaseous medicament can also be provided in a container as liquefied gas or gas mixture or in cryogenically solidified form.

The spasmolytic is usually administered using a ventilation machine with a gas-metering unit or with an anesthesia machine. The medicament is advantageously produced directly from the pure gases for use, for example by mixing xenon, oxygen and, where appropriate, an inert gas (for example with the aid of an anesthesia machine) in the direct vicinity of the patient.

The spasmolytic is administered as dry gas, moist gas or water vapor-saturated gas to the patient.

The spasmolytic of the invention, in particular the gaseous spasmolytic which is administered by inhalation, is also used in combination with a conventional spasmolytic which is administered in particular orally or intravenously. This allows inter alia better adjustment to the individual states of the patient's illness, with targeted use and dosing of the in some cases differing effects of the different spasmolytics so that the effects of the spasmolytics advantageously supplement one another. The spasmolytic of the invention and one or more conventional spasmolytics are accordingly used as combination medicament. The invention thus further relates to a spasmolytic comprising xenon or a xenon-containing gas and a spasmolytic which is administered orally, by inhalation or intravenously, preferably with an organic spasmolytic active ingredient, as combination product for simultaneous, separate or sequential use for the treatment of spasms, especially vasospasms. It is likewise advantageous to combine the xenon-containing spasmolytic with an NO source.

A nitric oxide source (NO source) is NO (nitric oxide), an NO-containing gas or gas mixture or a substance or preparation which releases nitric oxide (NO), which stimulates endogenous NO production or inhibits the breakdown of NO in the body. A nitric oxide source are in particular NO-releasing and/or NO-forming compounds.

NO sources such as NO-containing gas mixtures and NO-releasing compounds are described for example in DE 691 27 756 T2 (e.g. page 8, line 7, to page 9, end of the second paragraph, therein), which is incorporated herein by reference. Examples of NO-releasing compounds are S-nitroso-N-acetyl-penicillamine (SNAP), S-nitrosocysteine, nitroprusside, nitrosoguanidine, glycerol trinitrate, isoamyl nitrite, inorganic nitrite, azide or hydroxylamine. The NO-releasing compounds are introduced for example as aerosols through inhalation into the lung, as described in DE 691 27 756 T2, which is incorporated herein by reference.

Xenon and an NO source are advantageously administered for pulmonary complaints in combination via the lung, with the effects of xenon and NO supplementing one another. Medicaments, especially inhalable medicaments, with xenon and an NO source are generally for the treatment, prophylaxis or prevention of respiratory disorders, pulmonary function disorders, acute or chronic pulmonary hypertension, especially pneumonia, traumatic injury, aspiration or inhalation injury, fat embolism in the lung, acidosis, pneumonia, adult respiratory distress syndrome, acute pulmonary edema, asthma, pulmonary hypertension following cardiac surgery, persistent neonatal pulmonary hypertension, perinatal aspiration syndrome, hyaline membrane disease, acute pulmonary thromboembolism, heparin-protamine reactions, sepsis, chronic pulmonary hypertension, bronchopulmonary dysplasia, chronic pulmonary thromboembolism, idiopathic or primary pulmonary hypertension, IRDS (infant respiratory distress syndrome), asthma, PPH (innate pulmonary hypertension), cardiac malformation, lung immaturity in premature babies and neonates. Xenon and an NO source are also used to produce an inhalable medicament for the prevention, prophylaxis, treatment or after-treatment of apnea, especially following anesthesia.

Xenon and an NO source are particularly advantageously used to produce an inhalable medicament for the prevention, prophylaxis, treatment or after-treatment of apnea in premature babies and neonates, especially following anesthesia.

The invention thus relates to a medicament comprising xenon and an NO source. A medicament of this type consists for example of xenon and an NO source such as NO, of xenon, an NO source such as NO and an inert gas, or of xenon, an NO source such as NO, oxygen and an inert gas.

The invention further relates to a medicament, e.g. an inhaled medicament, preferably a spasmolytic, in particular a bronchospasmolytic, comprising xenon or a xenon-containing gas and an NO source as combination product for simultaneous, separate or sequential use, especially for pulmonary disorders in humans or mammals.

The gaseous medicaments are ordinarily used as a gas mixture which maintains respiration and which comprises xenon and oxygen or xenon, an NO source and oxygen. Gas mixtures of this type are employed for example in emergency medicine, where gas-mixing or gas-metering devices are too complicated for mobile use.

Examples of gases or gas mixtures which are employed as spasmolytic, in particular as bronchospasmolytic, are: 1.) 80% by volume xenon/x ppm NO/remainder oxygen; 2.) 70% by volume xenon/x ppm NO/remainder oxygen; 3.) 65% by volume xenon/5% by volume nitrogen/x ppm NO/remainder oxygen; 4.) 65% by volume xenon/x ppm NO/remainder oxygen; 5.) 60% by volume xenon/10% by volume nitrogen/x ppm NO/remainder oxygen; 6.) 60% by volume xenon/5% by volume nitrogen/x ppm NO/remainder oxygen; 7.) 60% by volume xenon/x ppm NO remainder oxygen; 8.) 55% by volume xenon/20% by volume nitrogen/x ppm NO/remainder oxygen; 9.) 55% by volume xenon/15% by volume nitrogen/x ppm NO/remainder oxygen; 10.) 55% by volume xenon/10% by volume nitrogen/x ppm NO/remainder oxygen; 11.) 55% by volume xenon/5% by volume nitrogen/x ppm NO/remainder oxygen; 12.) 55% by volume xenon/x ppm NO/remainder oxygen; 13.) 50% by volume xenon/x ppm NO/remainder oxygen; 14.) 50% by volume xenon/5% by volume nitrogen/x ppm NO/remainder oxygen; 15.) 50% by volume xenon/10% by volume nitrogen/x ppm NO/remainder oxygen; 16.) 50% by volume xenon/20% by volume nitrogen/x ppm NO/remainder oxygen; 17.) 50% by volume xenon/25% by volume nitrogen/x ppm NO/remainder oxygen; 18.) 45% by volume xenon/x ppm NO/remainder oxygen; 19.) 45% by volume xenon/5% by volume nitrogen/x ppm NO/remainder oxygen; 20.) 45% by volume xenon/10% by volume nitrogen/x ppm NO/remainder oxygen; 21.) 45% by volume xenon/15% by volume nitrogen/x ppm NO/remainder oxygen; 22.) 45% by volume xenon/20% by volume nitrogen/x ppm NO/remainder oxygen; 23.) 45% by volume xenon/25% by volume nitrogen/x ppm NO/remainder oxygen; 24.) 45% by volume xenon/25% by volume nitrogen/x ppm NO/remainder oxygen; 25.) 40% by volume xenon/30% by volume nitrogen/x ppm NO/remainder oxygen; 26.) 40% by volume xenon/10% by volume nitrogen/x ppm NO/remainder oxygen; 27.) 35% by volume xenon/40% by volume nitrogen/x ppm NO/remainder oxygen; 28.) 35% by volume xenon/x ppm NO/remainder oxygen; 29.) 30% by volume xenon/x ppm NO/remainder oxygen; 30.) 30% by volume xenon/20% by volume nitrogen/x ppm NO/remainder oxygen; 31.) 30% by volume xenon 40% by volume nitrogen/x ppm NO/remainder oxygen; 32.) 20% by volume xenon/x ppm NO/remainder oxygen; 33.) 20% by volume xenon/50% by volume nitrogen/x ppm NO/remainder oxygen; 34.) 15% by volume xenon/55% by volume nitrogen/x ppm NO/remainder oxygen; 35.) 15% by volume xenon/35% by volume nitrogen/x ppm NO/remainder oxygen; 36.) 10% by volume xenon/x ppm NO/remainder oxygen; 37.) 10% by volume xenon/40% by volume nitrogen/x ppm NO/remainder oxygen; 38.) 10% by volume xenon/60% by volume nitrogen/x ppm NO/remainder oxygen; 39.) 10% by volume xenon/65% by volume nitrogen/x ppm NO/remainder oxygen; 40.) 5% by volume xenon/70% by volume nitrogen/x ppm NO/remainder oxygen; 41.) 5% by volume xenon/65% by volume nitrogen/x ppm NO/remainder oxygen; 42.) 5% by volume xenon/45% by volume nitrogen/x ppm NO/remainder oxygen; 43.) 5% by volume xenon/65% by volume nitrogen/x ppm NO/remainder oxygen; 44.) 5% by volume xenon/x ppm NO/remainder oxygen; 45.) 1% by volume xenon/x ppm NO/remainder oxygen; 46.) 1% by volume xenon/69% by volume nitrogen/x ppm NO/remainder oxygen; 47.) 1% by volume xenon/74% by volume nitrogen/x ppm NO/remainder oxygen. The gas mixtures generally comprise from 0 to 100 ppm NO (x=0 to 100), preferably 0 to 50 ppm NO (x=0 to 50), particularly preferably 5 to 50 ppm NO (x=5 to 50), in particular 10 to 50 ppm NO (x=10 to 50). The gas mixtures are preferably produced close to the patient, for example from xenon gas, NO-inert gas mixture and oxygen or from xenon-NO gas mixture (e.g. xenon with 10 to 1000 ppm NO; in particular as compressed gas in compressed gas container) and oxygen.

Xenon is administered in particular during long-term ventilation and for prophylaxis in subanesthetic concentrations in a breathable gas (respiratory gas). It is advantageous especially during long-term ventilation to administer breathable gases having a content of from 5 to 45% by volume xenon, preferably 5 to 40% by volume xenon. During long-term ventilation, the breathable gas has, for example, a content of from 20 to 30% by volume oxygen, it being possible to increase the oxygen content if required at times for example 30 to 95% by volume oxygen. The remaining gas in the breathable gas usually consists of nitrogen or another inert gas and 0 to 100 ppm NO, preferably 0 to 40 ppm NO, particularly 5 to 40 ppm NO, in particular 5 to 20 ppm NO. The NO can also advantageously be added to the breathable gas only at times.

NO and NO-containing gases are preferably provided as compressed gases in pressurized containers, for example 200 to 800 ppm NO in nitrogen.

The gas mixtures with xenon and NO are preferably freshly produced close to the patient.

The gaseous medicament is usually administered using a ventilation machine with a gas-metering unit or with an anesthesia machine. The medicament is advantageously produced directly from the pure gases for use, for example by mixing xenon, oxygen and, where appropriate, an inert gas and an NO-containing gas (for example with the aid of an anesthesia machine or a gas-metering device) in the direct vicinity of the patient.

One, more than one or all of the gas components of the gaseous medicament, in particular xenon, NO-containing gas and oxygen or a respiratory gas, are advantageously mixed with the aid of a gas-metering device. The gas-metering device is used advantageously to vary the concentrations of the gas components during a ventilation. The device and the various methods for gas metering, in particular continuous and discontinuous gas metering with constant or variable gas component concentration, are described in DE 197 46 742 A1 and WO 98/31282, to which reference is hereby made.

The metering of one or more gases, in particular of NO, advantageously takes place only during the phases of breathing in (inspiration). No gas metering takes place during the breathing out (expiration). Gas metering synchronized with the breathing cycles is achieved by triggering with the aid of a sensor. A control unit identifies, on the basis of sensor measurements, the start of inspiration or the start and the end of expiration. The gas metering takes place continuously (e.g. with a previously fixed amount or concentration of the metered gas per inspiration over the entire operating time) or discontinuously (e.g. with metering intervals), preferably program-controlled (e.g. time program), sensor-controlled or with a combined program control and sensor control.

Metering of a gas, in particular xenon and/or NO-containing gas, advantageously takes place for many applications by combination of a basic metering and of an additive metering of one or more gases. Suitable as basic metering is, for example, a metering of xenon and NO-containing gas, a metering of xenon or a metering of NO-containing gas. As additive metering, for example, xenon and NO-containing gas, xenon or NO-containing gas is metered into a respiratory gas.

The use of one or more sensors on the patient permits automatic, patient-based metering of one or more gases.

1. A spasmolytic comprising xenon or a xenon-containing gas mixture.

2. The spasmolytic as claimed in claim 1, characterized in that xenon is present in therapeutically effective amount.

3. The spasmolytic as claimed in claim 1, characterized in that the spasmolytic comprises NO or an NO source in therapeutically effective amount.

4. A spasmolytic as claimed in claim 1, comprising xenon or a xenon-containing gas and a spasmolytic which is administered orally or intravenously as combination product for simultaneous, separate or sequential use in the treatment of spasms.

5. The spasmolytic as claimed in claim 4, characterized in that the spasmolytic is used for the treatment of vasospasms.

6. A medicament comprising xenon and an NO source or comprising a xenon-containing gas and an NO source.

7. The use of xenon or of a xenon-containing gas mixture according to claim 1, and, where appropriate, of an NO source to produce a medicament for the treatment of spasms, to produce a medicament for the treatment of vasospasms, to produce a medicament for the treatment of cerebral vasospasms, to produce a medicament for the treatment of impairments of blood flow, in particular to produce a gaseous medicament for the treatment of impairments of blood flow in the brain, to produce a medicament for the treatment of impairment of cerebral perfusion, to produce a medicament for the treatment of coronary perfusion impairment, to produce a medicament for the treatment of cognitive impairments, to produce a medicament for the treatment of stroke, to produce a medicament for the prophylaxis of stroke, to produce a medicament for improving the oxygen supply in the brain, to produce a medicament for the treatment von post-ischemia syndrome or to produce a medicament for promoting blood flow in the brain, to produce a medicament for improving the oxygenation of the body, to produce a medicament for improving the oxygenation of the brain.

8. The use of xenon or of a xenon-containing gas mixture according to claim 1, to produce a medicament having a vasodilating effect in capillary or precapillary vascular systems of the human body.

9. The use of xenon or of a xenon-containing gas mixture according to claim 1, and, where appropriate, of an NO source to produce a medicament for the prophylaxis and/or therapy of impairments of cognitive performance, also postoperatively.

10. The use of xenon or of a xenon-containing gas mixture according to claim 1, and, where appropriate, of an NO source for the treatment of spasms.

11. The use of xenon or of a xenon-containing gas, oxygen and, where appropriate, of an NO source for supplying oxygen to spontaneously breathing patients.
